(19) United States
(12) Patent Application Publication
(10) Pub. No.: US 2019/0126015 A1
(43) Pub. Date: May 2, 2019

(54) MEDICAL TUBE FIXING APPARATUS

(71) Applicant: ZIONMED CO., LTD., Seoul (KR)

(72) Inventor: Suk-Il Lee, Seoul (KR)

(73) Assignee: ZIONMED CO., LTD., Seoul (KR)

(21) Appl. No.: 16/214,396

(22) Filed: Dec. 10, 2018

Related U.S. Application Data
(63) Continuation-in-part of application No. 15/034,926, filed on May 6, 2016, now abandoned.

(30) Foreign Application Priority Data
Nov. 7, 2013 (KR) 10-2013-0134840

(51) Int. Cl. A61M 27/00; A61B 17/04; A61M 39/08; A61B 17/064; A61B 17/068; A61B 17/00

(52) U.S. Cl. CPC A61M 27/00; A61B 17/0469; A61M 39/08; A61B 17/064; A61B 17/08; A61B 17/00234; A61M 2039/087; A61M 2210/1003; A61B 17/068

(57) ABSTRACT

A medical tube fixing device and a skin stapler for securing a medical tube to an incised surgical site are disclosed. First and second fixing members of a body unit of the medical tube fixing device are loaded into the skin stapler, and can be closed by a pressure successively applied by the skin stapler to enclose and hold the medical tube. Simultaneously, a pair of fixing pins fixed to the body unit can be further bent to penetrate into and be fixed to the skin. Fixing the medical tube to the skin can be carried out by loading the medical tube fixing device on the skin stapler and pressurizing the skin stapler in a single-sequence pressing operation, which allows simple and quick fixing of the medical tube.

### CROSS-REFERENCE TO RELATED APPLICATION

This application is a continuation-in-part application of U.S. patent application Ser. No. 15/034,926 filed on May 6, 2016, which is a U.S. National Stage Application of International application No. PCT/KR2013/010099, filed on Nov. 8, 2013 and published in Korean as WO2015/068870 on May 14, 2015, and claims priority to and benefit of Korean Patent Application No. 10-2013-0134840, filed on Nov. 7, 2013 before the Korean Intellectual Property Office (KIPO), the disclosures of which are incorporated by reference in their entirety herein.

### BACKGROUND

### 1. Technical Field

The present invention relates to a medical tube fixing apparatus capable of securing a medical tube to the skin near an incised surgical site of a person or an animal (hereinafter, referred to as ‘a patient’).

### 2. Discussion of the Related Art

In general, body fluids such as blood, pus, or the like may be generated from organs of the patient and stagnate within the patient's body after surgery of the patient. Surgeons may use a drain device to quickly drain the body fluids out of the body for a quick recovery of the patient or animal.

The drain device may include a medical tube for discharging the body fluids outside the patient's body. There are widely used medical tubes such as a catheter that is used for discharging bile, abscess or the like from the liver, stomach or the like outside the human body, an uretera catheter that is inserted into a bladder and discharges urine, and a drainage tube, of which one end portion is inserted into a human body and the remaining portion is exposed to the outside, for discharging the body fluids generated from the internal organs outside the body, etc.

Recently a medical skin stapler has been widely used to seal the surgical site in a state that the medical tube is inserted into the patient's body after surgery of the patient. The skin stapler is a tool to improve the conventional method of suturing the skin incised by a surgical operation or a cosmetic surgery with a suture thread and a needle. This is, it is meant that the skin stapler is a device for suturing the incised skin using staples. Conventionally, the staple used by the skin stapler has a substantially ‘ [symbol] -shape’, and is loaded in a cartridge of the skin stapler. Both ends of the staple are bent inwards to suture the incised skin when the staple is pressed by the skin stapler. In a state that the incised skin is sutured like this, an exposed medical tube portion is fixed to the patient's body by using a conventional medical band or a method of directly binding it to the skin with a thread used.

However, adhesion of the medical band may become weakened due to long-time use and thus it should be frequently replaced. Gaps may be formed between the medical band and the medical tube whenever the patient moves, and thus an adhesive force by the medical band may be weakened and the medical tube may be pushed out to the outside.

In addition, there may be some other problems. The medical tube may be pushed out or the organs may be damaged due to insufficient adhesion of the medical band. The escape of the medical tube from the body may cause the pleura fluid, etc. to leak and cause the infections or the pulmonary emphysema due to air infiltration.

Furthermore, there is a method of binding the medical tube with a thread but it is not so robust and thus the medical tube inserted into the patient's body may easily escape from the body to the outside due to movement of the medical tube.

In consideration of these problems, there was a Korean Patent No. 10-1064844 entitled “An apparatus for fixing a medical tube”.

The Korean Patent No. 10-1064844 provides a fixing apparatus of the medical tube that is installed through the skin of the patient. The fixing apparatus includes a tape, a tube guiding member, and a fixing cap. The tube guiding member is formed with a tube through-hole for penetration of a tube at the center thereof and includes an adhesive layer formed on a bottom surface thereof to be attached to the skin of a patient. The tube guiding member includes a tape fixing member of which top surface or bottom surface is fixed to the vicinity of the tube through-hole, and a cap insertion member formed in a cylindrical shape on the tape fixing member, wherein a cap mounting hole is formed through the centers of the tape fixing member and the cap insertion member. The fixing cap is formed at its center with a guide hole through which the tube is inserted, and includes a supporting member formed in a plate type along a circumference of the guide hole. A plurality of pressing pieces of which outer circumference surfaces are downwardly slanted to the center are formed on a bottom portion of the supporting member and spaced apart from each other along the circumference of the guide hole. Inner surfaces of the pressing pieces tightly hold the tube, and outer surfaces of the pressing pieces inserted into the cap mounting hole of the tube guide member can be tightly gripped by the inner wall of the cap insertion member.

However, the apparatus for fixing the medical tube according to the prior art has some disadvantages. Its manufacturing cost is relatively high because its elements such as the fixing cap, the tube guide member and the tape should be manufactured separately, It takes lots of time to fix the medical tube fixing apparatus to the patient using the tape. As the apparatus for fixing the medical tube is used for a long time, a fixing force applied to the medical tube by the apparatus may be weakened and allow the medical tube to be loosened and to freely move.

In addition, the fixing apparatus may cause skin irritation because the tape is directly attached to the skin. When the fixing apparatus is used, it is directly attached to a patient's body and thus is used as a disposable one. When it is required that the apparatus for fixing the medical tube should be replaced, its entire replacement is needed, which results in high costs.

### SUMMARY

The present disclosure is to provide a medical tube fixing device that can be mounted on a skin stapler to be used for the purpose of quickly and easily holding and fixing a medical tube to skin near a surgical site by a single-sequence pressing operation with a skin stapler, and reduce a treatment fee by lowering production costs. In addition, the present disclosure is to provide a skin stapler which can be used for coupling the medical tube fixing device with the medical tube and fixing them to the skin by pressing a lever of the skin stapler in a single-sequence.

The present invention is to provide a medical tube fixing apparatus capable of quickly and easily holding the medical tube inserted into a surgical site of the skin and fixing the same to the skin near the surgical site by a single-sequence pressing operation. The various problems intended to be solved by the present disclosure are not limited to those mentioned above, and any other problems that are not mentioned above will be clearly understood by one of ordinary skill in the art from the following description.

A medical tube device according to exemplary embodiments of the present invention includes a body unit, and at least a pair of fixing pins. The body unit is configured to include first and second fixing members which are coupled to each other so as to be openable and closable and capable of enclosing and holding an outer surface of a medical tube in a hollow cylindrical shape while facing each other when being closed by a first external force; a non-slip member provided on an inner surface of at least one of the first and second fixing members and configured to enhance a frictional force with the outer surface of the medical tube; and a locking unit configured to lock the first and second fixing members to each other so that the first fixing member and the second fixing member do not open in a closed state. The at least a pair of fixing pins is configured to protrude outwardly from the body unit while being fixed to the body unit, to have a shape bent in a direction in which end parts thereof are closer to each other so that the pair of fixing pins are further bent by a second external force pressing them by a skin stapler to form a closed loop when end parts thereof come to meet each other.

In an embodiment of the present invention, the at least one pair of fixing pins may be configured to extend in both opposite directions substantially perpendicular to an axial direction of the body unit to provide a protruding portion outside the body unit, so that the at least one pair of fixing pins can receive the second external force directly from the skin stapler.

In an embodiment of the present invention, the medical tube fixing device may be intended to be used for a purpose that the first and second fixing members of the body unit are closed to enclose and hold the medical tube by a pressure successively applied by the skin stapler in a state that the medical tube fixing device is loaded on the skin stapler.

In an embodiment of the present invention, the body unit may be configured to have an inner diameter smaller than an outer diameter of the medical tube and to be in close contact with the outer surface of the medical tube.

In an embodiment of the present invention, the locking unit may include a first supporting plate formed with a through-hole and fixed to the first fixing member; and a second supporting plate formed with a protrusion to be press-fitted into the through-hole and fixed to the second fixing member.

In an embodiment of the present invention, the pair of fixing pins may be fixed to the first supporting plate and the second supporting plate, respectively.

In an embodiment of the present invention, the non-slip member may be any one of a plurality of elastic protrusions or elastic non-slip pads coated on at least a part of an inner surface of at least one of the first fixing member and the second fixing member.

A skin stapler according to exemplary embodiments of the present invention includes a housing, and elastic member, an actuator lever, a body tightening unit, a fixing pin tightening unit, a first pressure transmission mechanism, and a second pressure transmission mechanism. The elastic member is installed inside the housing. The actuator lever is configured to be a structure that a part of the actuator lever is exposed to an outside of the housing and the other part of the actuator lever is present inside the housing, and to be supported and pushed out of the housing by the elastic member while being caught by the housing so as not to escape to the outside of the housing. The body tightening unit is disposed outside the housing and configured to include a first gripping hand structure which is able to be pivotally closed around a first axis so as to enclose and apply a first force to an outer surfaces of an openable cylindrical body unit of a medical tube fixing device to be pinched and closed. The fixing pin tightening unit is disposed outside the housing and configured to include a second gripping hand structure which is able to be pivotally closed around a second axis so as to enclose and apply a second force to at least a pair of fixing pins of the medical tube fixing device so that distal ends of the pair of fixing pins come close to each other. The first pressure transmission mechanism is built in the housing and configured to receive a first pressure from the actuator lever and to convert the first pressure into the first force to pinch the body tightening unit and transmit the first force to the body tightening unit. The second pressure transmission mechanism is built in the housing and configured to receive a second pressure from the actuator lever and to convert the second pressure into the second force to pinch the fixing pin tightening unit and transmit the second force to the fixing pin tightening unit.

In an embodiment of the present invention, the skin stapler may be intended to be used in a manner that, when the body unit and the pair of fixing pins are respectively loaded on the body tightening unit and the fixing pin tightening unit, a force applied by a user to the actuator lever of the skin stapler is transferred to the body tightening unit and the fixing pin tightening unit to pinch the body unit and the pair of fixing pins for a purpose of coupling the medical tube fixing device with a medical tube and fixing the medical tube fixing device to skin.

In an embodiment of the present invention, the first pressure transmission mechanism may include a first pressing part provided at a distal end thereof and configured to press the body tightening unit to be pinched while being advanced by the first pressure.

In an embodiment of the present invention, the second pressure transmission mechanism may include a second pressing part provided at a distal end thereof and configured to press the fixing pin tightening unit to be pinched while being advanced by the second pressure.

A medical tube fixing apparatus according to exemplary embodiments of the present invention includes a medical tube fixing device, a skin stapler. The medical tube fixing device includes a body unit and at least a pair of fixing pins. The body unit is configured to include first and second fixing members which are coupled to each other so as to be openable and closable and capable of enclosing and holding an outer surface of a medical tube in a hollow cylindrical shape while facing each other when being closed by a first external force; a non-slip member provided on an inner surface of at least one of the first and second fixing members and configured to enhance a frictional force with the outer surface of the medical tube; and a locking unit configured to lock the first and second fixing members to each other so that the first fixing member and the second fixing member do not open in a closed state. The at least a pair of fixing pins is configured to protrude outwardly from the body unit while being fixed to the body unit, to have a shape bent in a direction in which end parts thereof are closer to each other so that the pair of fixing pins are further bent by a second external force pressing them by a skin stapler to form a closed loop when end parts thereof come to meet each other. The skin stapler include a housing, and elastic member, an actuator lever, a body tightening unit, a fixing pin tightening unit, a first pressure transmission mechanism, and a second pressure transmission mechanism. The elastic member is installed inside the housing. The actuator lever is configured to be a structure that a part of the actuator lever is exposed to an outside of the housing and the other part of the actuator lever is present inside the housing, and to be supported and pushed out of the housing by the elastic member while being caught by the housing so as not to escape to the outside of the housing. The body tightening unit is disposed outside the housing and configured to include a first gripping hand structure which is able to be pivotally closed around a first axis so as to enclose and apply a first force to an outer surfaces of an openable cylindrical body unit of a medical tube fixing device to be pinched and closed. The fixing pin tightening unit is disposed outside the housing and configured to include a second gripping hand structure which is able to be pivotally closed around a second axis so as to enclose and apply a second force to at least a pair of fixing pins of the medical tube fixing device so that distal ends of the pair of fixing pins come close to each other. The first pressure transmission mechanism is built in the housing and configured to receive a first pressure from the actuator lever and to convert the first pressure into the first force to pinch the body tightening unit and transmit the first force to the body tightening unit. The second pressure transmission mechanism is built in the housing and configured to receive a second pressure from the actuator lever and to convert the second pressure into the second force to pinch the fixing pin tightening unit and transmit the second force to the fixing pin tightening unit. The medical tube fixing apparatus is intended to be used in a manner that, when the medical tube fixing device id loaded on the skin stapler, the skin stapler presses the body unit and the pair of fixing pins by the body tightening unit and the fixing pin tightening unit to pinch the body unit and the pair of fixing pins for a purpose of coupling the medical tube fixing device with a medical tube and fixing the medical tube fixing device to skin.

In an embodiment of the present invention, the non-slip member may be any one of a plurality of elastic protrusions or elastic non-slip pads coated on at least a part of an inner surface of at least one of the first fixing member and the second fixing member.

In an embodiment of the present invention, the at least one pair of fixing pins may be configured to extend in both opposite directions substantially perpendicular to an axial direction of the body unit to provide a protruding portion outside the body unit, so that the at least one pair of fixing pins can receive the second external force directly from the skin stapler.

In an embodiment of the present invention, the first pressure transmission mechanism may include a first pressing part provided at a distal end thereof and configured to press the body tightening unit to be pinched while being advanced by the first pressure.

In an embodiment of the present invention, the second pressure transmission mechanism may include a second pressing part provided at a distal end thereof and configured to press the fixing pin tightening unit to be pinched while being advanced by the second pressure.

Specific details of other embodiments are included in the detailed description and drawings below.

The medical tube fixing fixture according to the present invention is a structure in which the body unit for holding the medical tube and the fixing pins for fixing to the patient's skin are integrally combined. The medical tube fixing device is a structure that can be simply and easily loaded on the skin stapler.

Further, the medical tube fixing apparatus according to the present invention can hold the medical tube and fix it to the patient's skin by pressing the actuator lever of the skin stapler in a single-sequence (one-touch) pressing operation. Therefore, the fixing operation of the medical tube can be performed easily and quickly. Since it is easy to manufacture the medical tube fixing apparatus, the manufacturing cost can be reduced, thereby reducing the medical cost burden on the patient.

In addition, the medical tube fixing device according to the exemplary embodiment of the present disclosure is capable of preventing contact dermatitis which may be caused by the adhesive having a large area, and removing the patient's inconvenience for movement as the medical tube fixing device can be produced in a significantly small size.

It may be fully understood that various exemplary embodiments of the technical spirit of the present disclosure may provide a variety of effects that are not specifically mentioned here.

### BRIEF DESCRIPTION OF THE DRAWINGS

FIGS. 1 and 2 illustrate perspective views of a medical tube fixing device according to an exemplary embodiment of the present disclosure.

FIG. 3 illustrates a cross-sectional view of the medical tube fixing device according to the exemplary embodiment of the present disclosure.

FIG. 4 illustrates a state in which the medical tube fixing device is used according to an exemplary embodiment of the present disclosure.

FIGS. 5 and 6 illustrate a state in which the medical tube fixing device is used in accordance with an exemplary other embodiment of the present disclosure.

FIGS. 7 and 8 illustrate a medical tube fixing device according to other exemplary embodiment of the present disclosure.

FIG. 9 illustrates a skin stapler according to another exemplary embodiment of the present disclosure.

FIGS. 10A-10D are views for describing a method of fixing the medical tube to the medical tube fixing device shown in FIG. 1 using the skin stapler shown in FIG. 9.

FIGS. 11A and 11B show an open state and a closed state of a medical tube fixing device according to other exemplary embodiment of the present disclosure, respectively.

FIGS. 12A to 16 illustrate a process of fixing the medical tube to the skin near a surgical site using a medical tube fixing apparatus including a skin stapler and the medical tube fixing device shown in FIGS. 9, 11A and 11B, respectively.

### DETAILED DESCRIPTION OF THE EMBODIMENTS

Advantages and features of the present inventive concept and methods of accomplishing the same will become apparent with reference to the embodiments that will be described below in detail, along with the accompanying drawings. The present inventive concept may, however, be embodied in many different forms and should not be construed as limited to the exemplary embodiments set forth herein. Rather, these exemplary embodiments are provided so that this disclosure will be thorough and complete, and will fully convey the scope of the present inventive concept to those skilled in the art. In the drawings, the sizes and relative sizes of layers and regions may be exaggerated for clarity.

It will be understood that, although the terms first, second, etc. may be used herein to describe various elements, these elements should not be limited by these terms. These terms are used just to distinguish one element from another. Thus, a first element may be termed a second element without departing from the teachings of the present inventive concept, and similarly the second element may be termed the first element.

The terminology such as the upper part, the lower part, the top surface, the top, the bottom, etc. will be used to distinguish between the relative positions of the components. For example, in case of naming the part above and the part below the middle of a drawing as the upper part and the lower part, in practice without departing from the scope of the present inventive concept the upper part may be referred to as the lower part, and the lower part may be referred to as the upper part.

The terminologies used herein are for the purpose of describing particular exemplary embodiments only and are not intended to be limiting of the present inventive concept. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms, including technical and scientific terms, used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

With reference to the accompanying drawings, preferred embodiments of the medical tube fixing device and apparatus according to the present inventive concept will be described in detail.

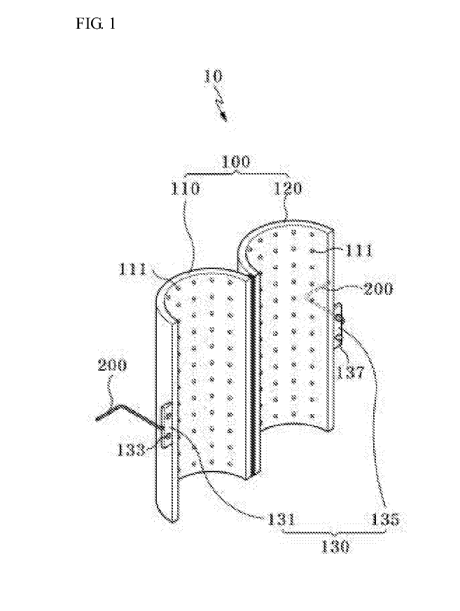

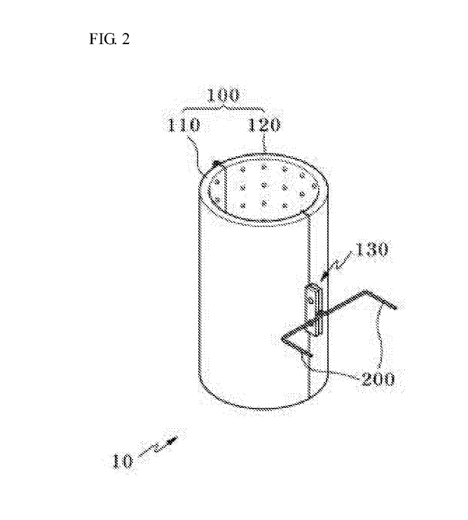

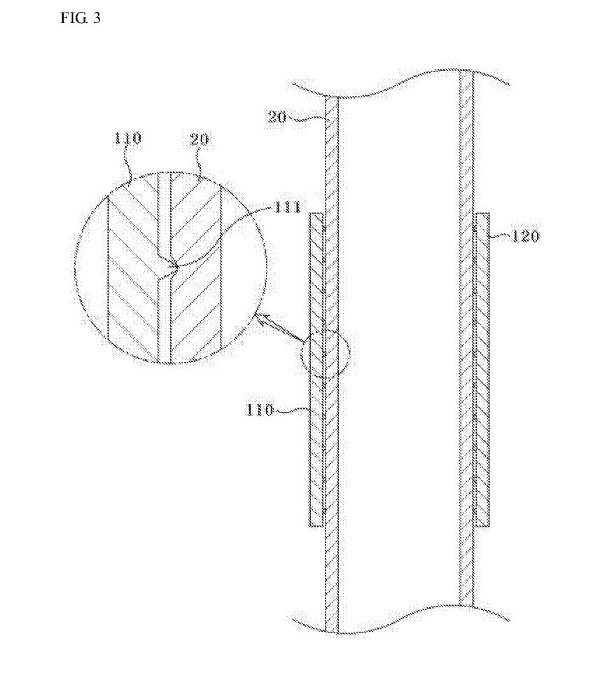

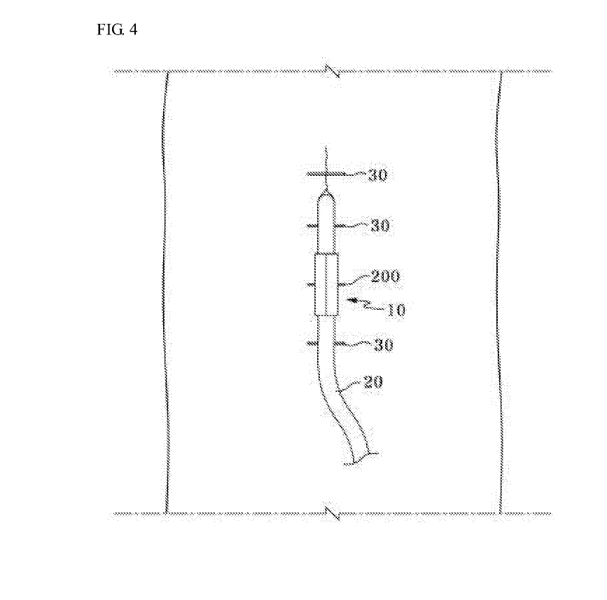

FIGS. 1 and 2 are perspective views of the medical tube fixing device according to an exemplary embodiment of the present disclosure. FIG. 3 is a cross-sectional view of the medical tube fixing device according to the exemplary embodiment of the present disclosure. FIG. 4 illustrates a state in which the medical tube fixing device is used according to an exemplary embodiment of the present disclosure.

With reference to FIGS. 1 to 4, the medical tube fixing device 10 is a device for holding and fixing firmly a medical tube 20 which is inserted into the patient's body and used for draining out quickly body fluids such as blood, pus, or the like which may stagnate within a patient's body after surgery.

The medical tube fixing device 10 may include a body unit 100 and a fixing pin unit 200.

The body unit 100 may be provided so as to hold and fix the medical tube 20 to the skin of the patient. The body unit 100 may be formed in a cylindrical shell shape of which inside is vacant, and its inner diameter may be slightly smaller than the outer diameter of the medical tube 20. The inner side of the body unit 100 may be made of rubber or silicone so as to firmly grip the medical tube 20 without slip. The body unit 100 may include two semi-cylindrical members, where a first fixing member 110 and a second fixing member 120 may be obtained by dividing a cylindrical shell into two half-pieces in its axial direction. A pair of lateral ends (referred to as ‘a first end’ and ‘a second end’) of the two semi-cylindrical members are connected to each other, and a pair of the other lateral ends of them are not connected to each other so that any one of the two semi-cylindrical members can be pivoted around the other in a circumferential direction, that is, opened and closed around the first lateral ends of them. The body unit 100 may further include a locking unit 130.

Each of the first and second fixing members 110 and 120 may be formed with a number of protrusions 111 configured to provide an emboss-effect. The protrusions may be spaced apart from each other by a predetermined interval on the inner side (wall) of the first fixing member 110.

The locking unit 130 may be provided to lock the first fixing member 110 and the second fixing member 120 together. The locking unit 130 may include a first supporting plate 131 and a second supporting plate 135.

The first supporting plate 131 may be fixed to the first end of the first fixing member 110 by its one end and extended outward to be a rectangular plate, for example. The first supporting plate 131 may be formed with one or more through-holes 133.

The second supporting plate 135 may be fixed to the second end of the second fixing member 120 by its one end and extended outwardly to be a rectangular plate, for example. The second supporting plate 135 may be provided with one or more protrusions 137 that can be press-fitted into the through-holes 133. The second supporting plate 135 may be coupled to the first supporting plate 131 by inserting the one or more protrusions 137 into the one or more through-holes 133.

The first supporting plate 131 and the second supporting plate 135 may be coupled to each other by being simply pressurized just one time with a skin stapler.

The locking unit 130 may have any other structure and/or element if it can allow the first fixing member 110 and the second fixing member 120 to be coupled and fastened to each other. For example, the locking unit 130 may be formed with a male member and a female member both of which can be fastened to each other in the manner of simply pressurizing.

The fixing pin unit 200 may include at least a pair of fixing pins. In an exemplary embodiment, a pair of fixing pins 200 may be fixed to both of the first fixing member 110 and the second fixing member 120 respectively, and inserted into and fixed to the skin of the patient in a manner of simple single-sequence pressurizing with a skin stapler 500. The pair of fixing pins 200 may be fixed to the first supporting plate 131 and the second supporting plate 135, respectively and at least partially protrude outward from the body unit 100. In other words, the pair of fixing pins 200 may be fixed to the first fixing member 110 and the second fixing member 120, respectively, or the first supporting plate 131 and the second supporting plate 135, respectively, relaying on the structure in which the locking unit 130 is formed. The pair of fixing pins 200 may have protruding portions which extend in both directions substantially perpendicular to the axial direction of the body unit 100 and beyond the body unit 100. And an external pressing force by the skin stapler 500 can be directly transmitted to the protruding portions of the pair of fixing pins 200. The distal ends of the pair of fixing pins 200 may be bent in a direction closer to each other by the external pressing force. That is, the pair of fixing pins 200 may be bent by the external pressing force, and the distal ends of the pair of fixing pins 200 can form an almost closed loop as they approach each other.

In the exemplary embodiment of the present disclosure, the medical tube fixing device 10 may be used for the purpose of holding the medical tube 20 inserted inside the incised skin of a surgical patient and fixing it securely to the skin as illustrated in FIG. 4. But this is an example. It will be understood by those skilled in the art that various modifications and variations can be made in the medical tube fixing device 10 according to the present invention.

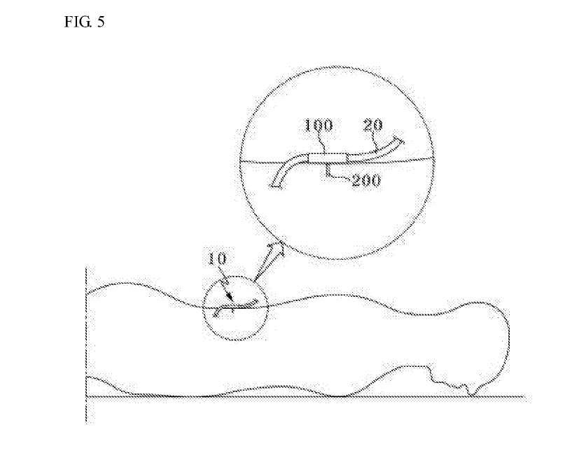

With reference to FIGS. 5 and 6, after a surgical site of the patient is sutured, another site on the skin around the surgical site may be incised to fix the medical tube 20 with the medical tube fixing device 10. For example, as shown in FIG. 6 (a) the surgical site may be sutured in accordance with the type of surgery, and as shown in FIG. 6 (b) the body fluids such as blood, pus, or the like which may stagnate within the patient's body may be drained out quickly through the incised another site spaced apart from the sutured surgical site by a predetermined distance a. In this case, as shown in FIG. 5 after the surgical site is sutured and the medical tube 20 is inserted into the another site around the surgical site, the body unit 100 can be firmly coupled with the medical tube 20 and the pair of fixing pins 200 can be inserted into and fixed to the patient's skin in the manner of the single-sequence pressuring of the skin stapler, that is, by simply pressurizing with the skin stapler.

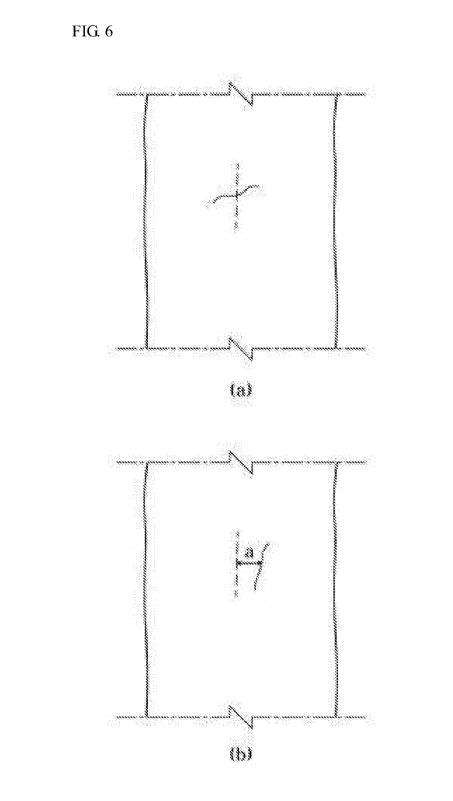

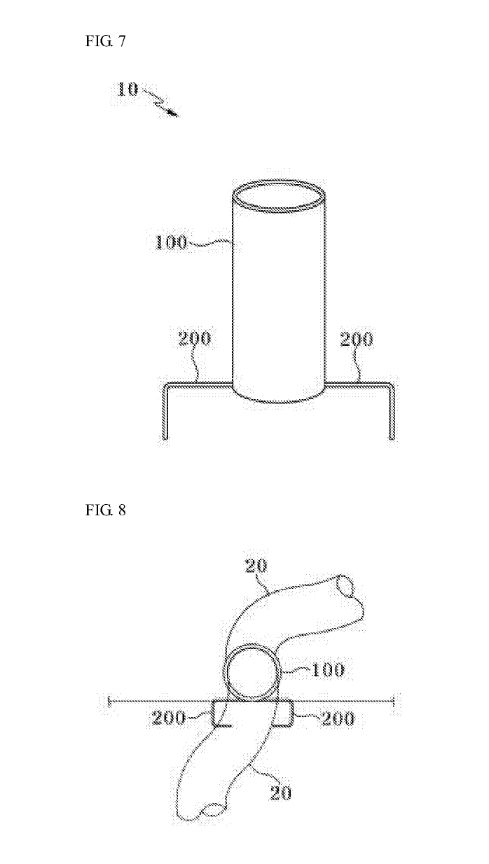

Also, various modifications of the medical tube fixing device 10 according to the present disclosure can be made as shown in FIGS. 7 and 8. For example, as shown in FIG. 7 the pair of fixing pins 200 may be fixed to a position of the body unit 100, and end portions of the pair of fixing pins 200 may be inserted into the patient's skin such that they can be bent.

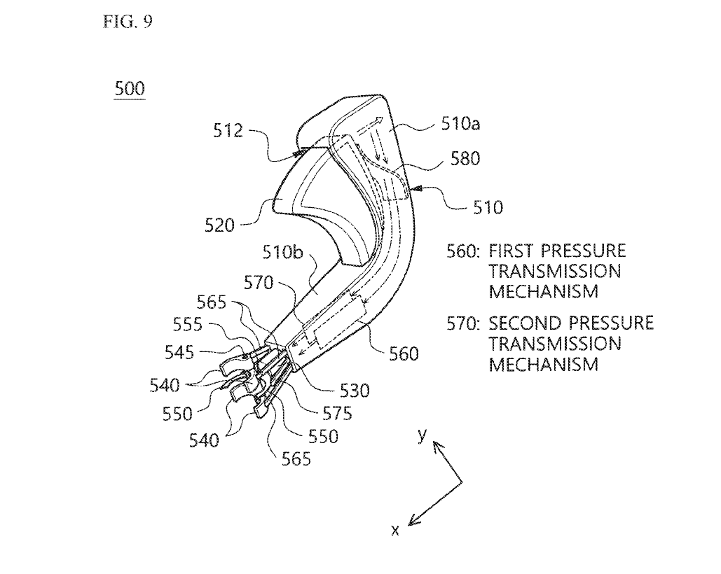

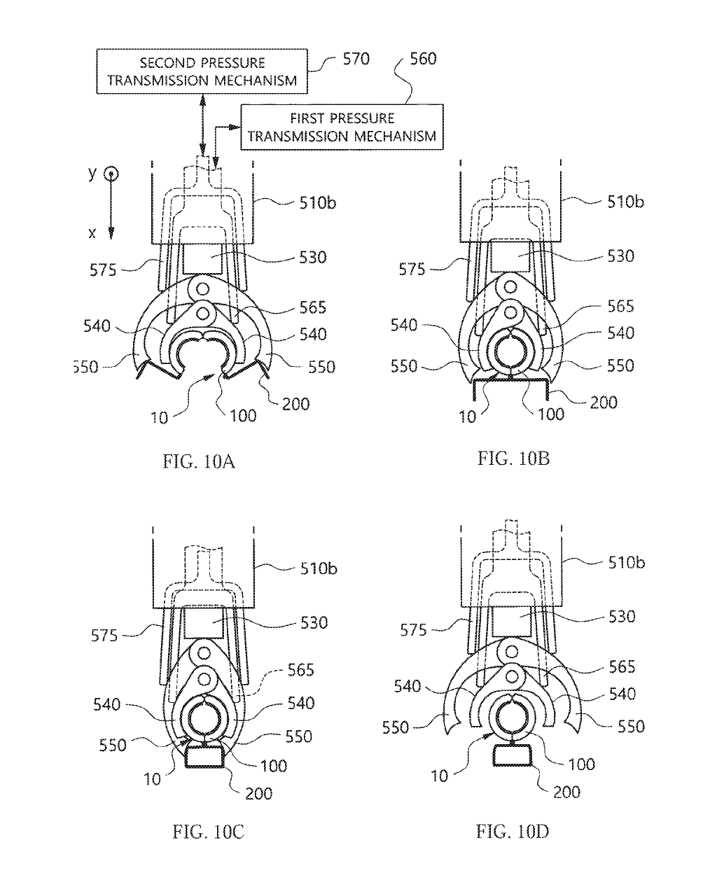

FIG. 9 illustrates a skin stapler 500 in accordance with an exemplary embodiment of the present disclosure. FIGS. 10A and 10B illustrate a process in which the skin stapler 500 shown in FIG. 9 presses the body 100 and the pair of fixing pins 200 of the medical tube fixing device 10.

Hereinafter, with reference to FIGS. 1 to 4 and FIGS. 9 and 10A-10D described in more detail is a fixing method of suturing the surgical site of the patient with the medical tube fixing device 10 with being loaded in the skin stapler 500.

Here, the skin stapler 500 is a means configured to fix the pair of fixing pins 200 to the skin of the patient while the body unit 100 of the medical tube fixing device 10 holds the medical tube 20.

With reference to FIGS. 9 and 10A-10D, the skin stapler 500 may include a handle housing 510, an actuator lever 520, a connecting portion 530, a body tightening unit 540, a fixing pin tightening unit 550, and first and second pressure transmission mechanisms 560 and 570.

According to an exemplary embodiment, the handle housing 510 may have a shape similar to a handgun, for example, and include a handle portion 510a and an extended barrel portion 510b extending generally perpendicular to the top of the handle 510a. The handle portion 510a of the handle housing 510 may be provided with an opening 512, and the end of the extended barrel portion 510b may be open.

The actuator lever 520 may be mounted through the opening 512 provided in the handle 510a of the handle housing 510. A part of the actuator lever 520 may be exposed to the outside of the handle housing 510 and the remaining part of the actuator lever 520 may be positioned inside the handle 510a of the handle housing 510 to be caught by the handle housing 510 so as not to come out of the handle housing 510. An elastic member 580 may be interposed between the actuator lever 520 and the inner wall of the handle housing 510. The elastic member 580 can apply a force to push the actuator lever 520 out of the handle portion 510a of the handle housing 510. Thus, when an external force for pushing the actuator lever 520 into the handle portion 510a of the handle housing 510 is not applied, the actuator lever 520 is pushed-out farthest away from the handle portion 510a of the handle housing 510 by the elastic member 580.

In an exemplary embodiment, the connecting portion 530 may connect the body tightening unit 540 and the fixing pin tightening unit 550 to a distal end of the extension portion of the handle housing 510. The body tightening unit 540 may be a gripping hand structure that can be pivotally coupled to the shaft 545 so as to be pivotally opened and closed about the shaft 545. As shown, the gripping hands of the body tightening unit 540 may include an upper gripping hand 540a and a lower gripping hand 540b spaced apart by a predetermined distance. The fixing pin tightening unit 550 may be also a gripping hand structure that can be pivotally coupled to the shaft 555 so as to be pivotally opened and closed. The gripping hand of the fixing pin tightening unit 550 may be disposed between the upper gripping hand 540a and the lower gripping hand 540b of the body tightening unit 540.

The first and second pressure transmission mechanisms 560 and 570 may be installed inside the handle housing 510. The first pressure transmission mechanism 560 may be provided between the actuator lever 520 and the body tightening unit 540. The first pressure transmission mechanism 560 may receive a first pressure applied to the actuator lever 520 and convert the first pressure into a force for tightening the body tightening unit 540. The first pressure transmission mechanism 560 may transmit the force to the body tightening unit 540. The first pressure may be a pressure that is obtained when a user pushes the actuator lever 520 approximately halfway into the housing 500. In an exemplary embodiment, the first pressure transmission mechanism 560 may include a first pressing part 565 provided at an distal end thereof. The first pressing part 565 may have a U-shaped structure in which its inlet is opened to allow the body tightening unit 540 to enter into the first pressing part 565. In a process that the body tightening unit 540 enters the first pressing part 565 by a relative movement, the first pressing part 565 can apply a pressure to the outer side surfaces of the body tightening unit 540 to make the gripping hand of the body tightening unit 540 become narrower so as to clamp the body unit 100.

The second pressure transmission mechanism 570 may be provided between the actuator lever 520 and the fixing pin tightening unit 550. The second pressure transmission mechanism 570 may receive a second pressure applied to the actuator lever 520 and convert the second pressure into a force for tightening the fixing pin tightening unit 550. The second pressure transmission mechanism 570 may transmit the force to the fixing pin tightening unit 550. The second pressure may be a pressure that is obtained when the user further pushes the actuator lever 520 into the housing 500 approximately by the other halfway in a state that the first pressure is obtained by pressing the actuator lever 520 into the housing 500 approximately halfway. In an exemplary embodiment, the second pressure transmission mechanism 570 may also include a second pressing part 575 provided at an distal end thereof. The second pressing part 575 may have a U-shaped structure in which its inlet is opened to allow the fixing pin tightening unit 550 to enter into the second pressing part 575. The second pressing part 575 presses the outer side surfaces of the fixing pin tightening unit 550 in the process of the fixing pin tightening unit 550 entering into the second pressing part 575 by the relative movement. The fixing pin tightening unit 550 can be tightened to close. In a process that the fixing pin tightening unit 550 enters the second pressing part 575 by a relative movement, the second pressing part 575 can apply a pressure to the outer side surfaces of the fixing pin tightening unit 550 to make the gripping hand of the fixing pin tightening unit 540 become narrower so as to vend the fixing pins 200.

Next, a method of fixing the medical tube fixing device 10 near the surgical site of the patient using the skin stapler 500 will be described.

First, the medical tube 20 may be inserted into the patient's body to drain out the blood, pus, or the like collected within the surgical site.

In the state that the medical tube is inserted into the patient's body, the surgical practitioner sutures the surgical site with a conventional skin stapler by pressurizing so as to eject the staples 30 and to seal the incised surgical site. In FIGS. 1 to 4, it is described as an example to suture the incised surgical site of the patient in the state that the medical tube 20 is inserted into the incised surgical site, but the present disclosure is not limited to this example. One of ordinary skill in the art may insert the medical tube 20 into another site of the skin spaced apart from the incised surgical site of the patient as illustrated in FIGS. 5 and 6.

Next, the medical tube 20 may be mounted on the skin stapler 500 according to an exemplary embodiment of the present invention. The body unit 100 of the medical tube fixing device 10 can be coupled to the medical tube 20 by the first halfway pressing of the skin stapler 500. This will be described in more detail with reference to FIGS. 9 and 10A-10D. When the first pressure to press the actuator lever 520 is applied, the first pressure transmission mechanism 560 receives the pressure and converts it into a force for making the first pressing part 565 move forward. The first pressing part 565 advances by a predetermined distance in the longitudinal direction (x-direction in FIGS. 9 and 10A-10D) of the extended barrel portion 510b, and thus pressurizes the outer sides of the body tightening unit 540. Accordingly, the gripping hands of the body tightening unit 540 in the open state are pressurized to be closed and the body unit 100 is clamped by the gripping hands of the body tightening unit 540 so as to be closed (refer to FIGS. 10A and 10B).

Then, the skin stapler 500 may be further pressurized (secondary halfway pressing) in order to fix the medical tube fixing device 10 to the patient's skin. Here, the secondary halfway pressing means that further pressure is applied to the actuator lever 520 of the skin stapler 500 which is in a pressurized state through the first halfway pressing.

As such, when the secondary pressuring is applied to the skin stapler 500, the fixing pins 200 of the medical tube fixing device 10 are inserted into the skin of the patient and bent so as to be fixed to the skin. Hereby the surgery may be completed.

This process will be described in detail with reference to FIGS. 9 and 10A-10D. When the secondary pressure that presses the actuator lever 520 is further applied, the second pressure transmission mechanism 570 may receive the pressure. Then, the second pressure transmission mechanism 570 may convert the pressure to a force for moving forward the second pressing part 575 and transmit the force to the second pressing part 575.

By the force, the second pressing part 575 may advance by a predetermined distance in the longitudinal direction (i.e., the x-direction) of the extended barrel portion 510b to push the outer surfaces of the fixing pin tightening unit 550. Accordingly, the fixing pin tightening unit 550 in an open state may be closed so that the pair of fixing pins 200 may be pressed to be closed (see FIG. 10C). In this state, when the pressure applied to the actuator lever 520 is completely removed, the actuator lever 520 may be returned to its original position by a recovery force applied by the elastic member 580. The first pressing part 565 and the second pushing part 575 may move back in the −x axis direction by the distance having advanced in the x-axis direction. Then, the body tightening unit 540 and the fixing pin tightening unit 550 may be restored to the open state (see FIG. 10D).

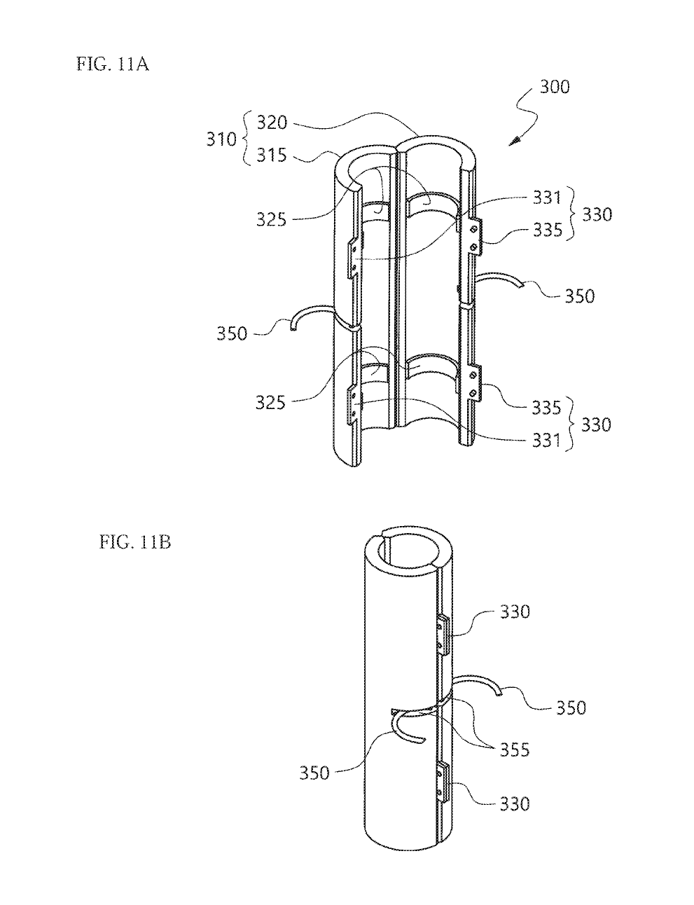

FIGS. 11A and 11B show the open state and the closed state of the medical tube fixing device 300 according to another exemplary embodiment of the present invention, respectively.

Referring to FIGS. 11A and 11B, the medical tube fixing device 300 may include a body unit 310, a locking unit 330, and a fixing pin unit 350 that includes at least a pair of fixing pins.

The body unit 310 may include a first fixing member 315 and a second fixing member 320, as in the body unit 100 described above. The first fixing member 315 and the second fixing member 320 may have a pair of vertical ends that are connected to each other along the axial direction and the other pair of vertical ends that are free. The first fixing member 315 and the second fixing member 320 may be opened or closed in the circumferential direction about their connected vertical ends. The first fixing member 315 and the second fixing member 320 may have a hollow cylindrical shape in a closed state. Some or all of the inner surface of the first fixing member 315 and the second fixing member 320 may be provided with one or more non-slip pads 325 which are brought into contact with the medical tube 20 so as not to slip. The non-slip pads 325 may be made of a material having good elasticity and frictional force, such as silicone, rubber, or the like.

Each of the first fixing member 315 and the second fixing member 320 may have a slot 355 formed along the circumferential direction at a position where the pair of fixing pins 350 are fixed. These slots 355 may be provided as a space in which the fixing pins 350 can be bent.

The locking unit 330 may have substantially the same configuration and function as the locking unit 130. That is, a plurality of locking units 330 may be provided, and each locking unit 330 may be fixed to each vertical end portion of the first fixing member 315 and the second fixing member 320. The locking unit 330 may include a first supporting plate 331 having through holes and a second supporting plate 335 having a fitting protrusion.

In an exemplary embodiment, the fixing pin unit 350 may include two fixing pins that are bent in an arc shape, for example. The two fixing pins 350 may be fixed to the outer surfaces of the first fixing member 315 and the second fixing member 320, respectively. The two fixing pins 350 may extend in both outward directions substantially perpendicular to the axial direction of the body unit 310 so that the two fixing pins 350 may have protrusion portions, respectively, that extend outside the body unit 310. The distal ends of the two fixing pins 350 may be bent downward (toward the circumferential direction). According to the fixing pins 350 having such a shape, an external pressing force by the skin stapler 500 may be directly transmitted to the protruding portions. The fixing pins 350 may be further bent downward by the external force, and can easily penetrate into the patient's skin. The shape of the fixing pins 350 shown is merely an example. The fixing pins 350 may have any other bent shape, for example, a round or angular shape.

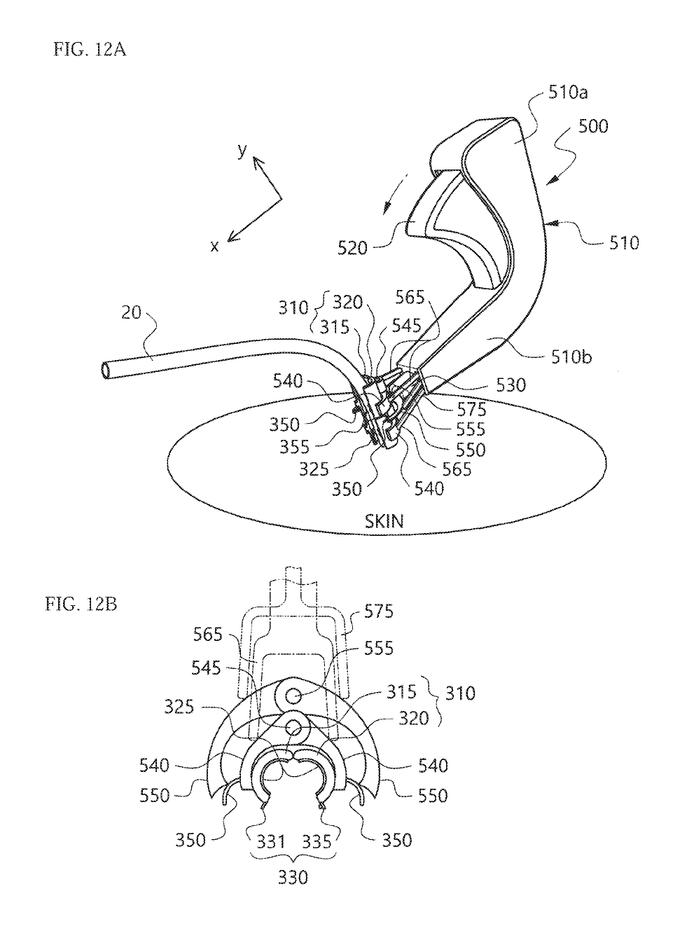

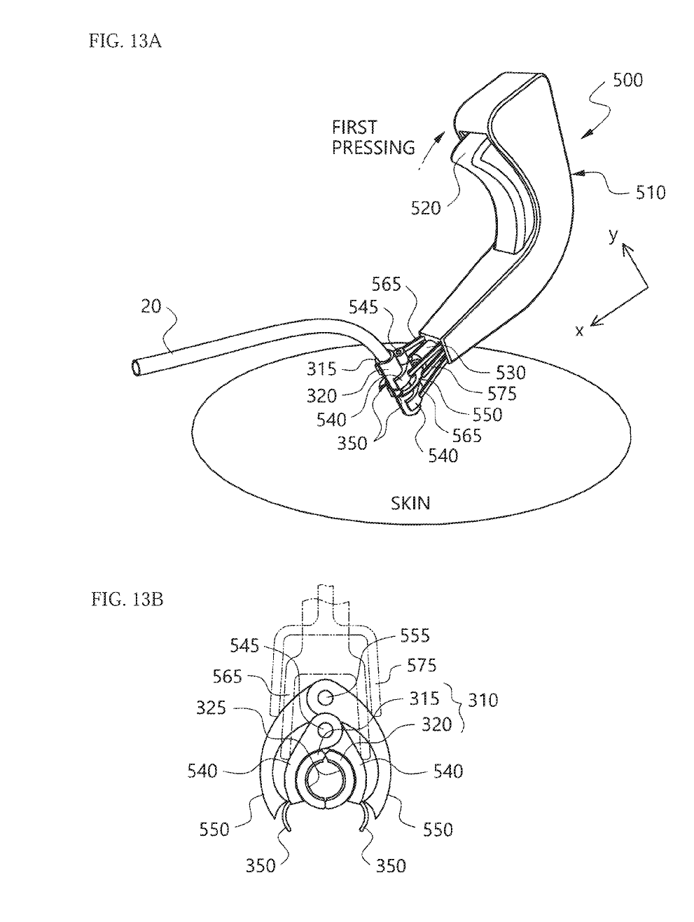

FIGS. 12A to 16 illustrate a process of fixing the medical tube 20 using a medical tube fixing apparatus that includes the skin stapler 500 and the medical tube fixing device 300 shown in FIGS. 9, 11A and 11B, respectively.

FIG. 12A shows a state in which the medical tube 20 and the medical tube fixing device 300 are loaded on the skin stapler 500. FIG. 12B is a cross-sectional view of an end portion the skin stapler 500 when viewed in a y-axis direction in this state. The illustrated state is that the medical tube 20 and the medical tube fixing device 300 are merely mounted on the skin stapler 500 and the actuator lever 520 is not yet under pressure. The first fixing member 315 and the second fixing member 320 of the medical tube fixing device 300 are positioned in an open state in the body tightening unit 540. In this state, the first pressing part 565 does not press the first and second fixing members 315 and 320, and the second pressing part 575 also does not press the fixing pin unit 350.

FIG. 13A shows a state in which a first pressure is applied to the actuator lever 520 of the skin stapler 500 and FIG. 13B is a cross-sectional view of an end portion the skin stapler 500 when viewed in a y-axis direction in this state. When the first pressure is applied to the actuator lever 520, the first pressing part 565 move forward by the first pressure transferred through the first pressure transmission mechanism 560. The body tightening unit 540 is pinched, which makes the body unit 310 of the medical tube fixing device 300 be pressed to be closed. When the first pressure is further applied, the second pressing part 575 may be further slightly advanced by the second pressure transmission mechanism 570. As a result, the fixing pin tightening unit 550 may become also narrower, so that the pair of fixing pins 350 of the medical tube fixing device 300 may be slightly further narrower.

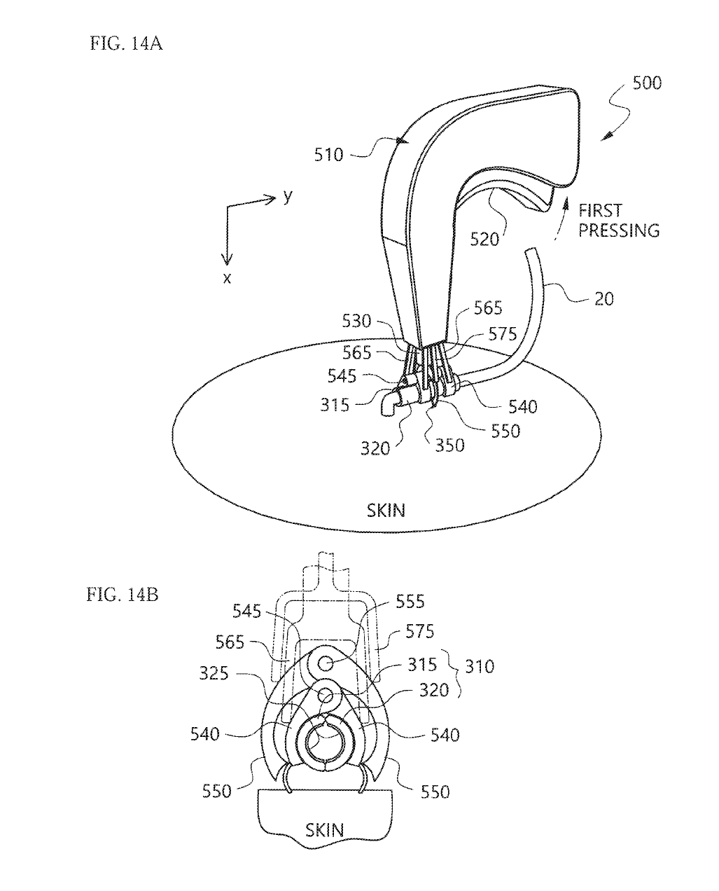

FIG. 14A shows a state in which the fixing pin 350 closely approaches the skin, and FIG. 14B is a cross-sectional view of an end portion the skin stapler 500 when viewed in a y-axis direction in this state. In the state shown in FIGS. 13A and 13B, the user may move the skin stapler 500 so that the pair of fixing pins 350 approach and contact with the skin as shown in FIGS. 14A and 14B. If the user further presses the actuator lever 520 of the skin stapler 500 in the state that the first pressure is being applied to the actuator lever 520, the pair of fixing pins 350 can penetrate the skin slightly.

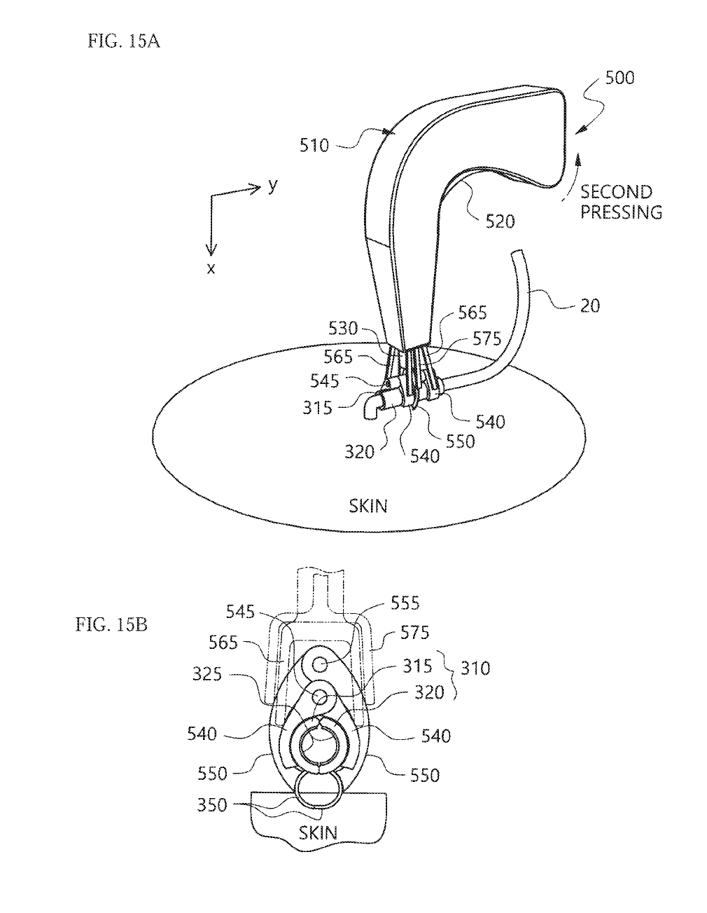

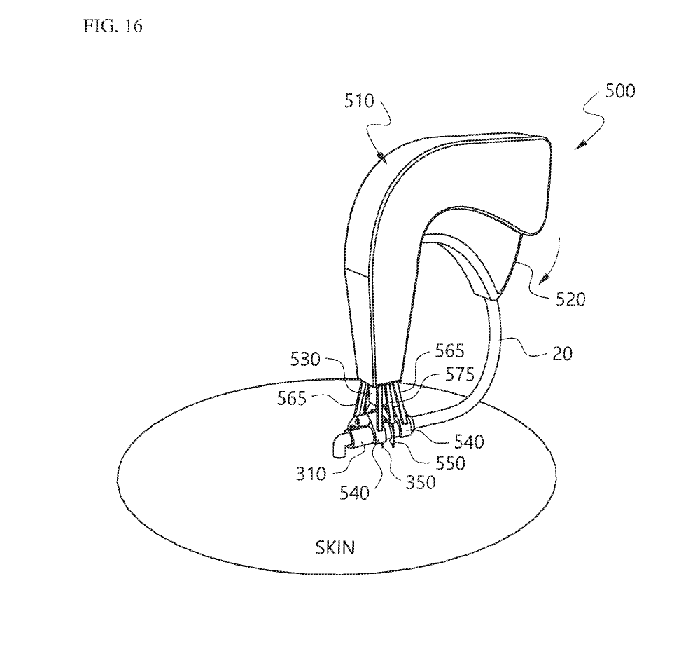

FIG. 15A shows a state in which a secondary pressure is applied to the actuator lever 520 of the skin stapler 500, and FIG. 15B is a cross-sectional view of an end portion the skin stapler 500 when viewed in a y-axis direction in this state. When the user additionally applies the secondary pressure to the actuator lever 520 in the state shown in

FIGS. 14A and 14B, the second pressing part 575 advances by the secondary pressure transferred through the second pressure transmission mechanism 570. Accordingly, the fixing pin tightening unit 550 may be pushed and press the pair of fixing pins 350 of the medical tube fixing device 300. As a result, both ends of the fixing pins 350 come close to abut each other. Thereby, the fixing pins 350 can be fixed to the skin, and as a result, the medical tube fixing device 300 can fix the medical tube 20. Here, the first pressure and the secondary pressure are pressures that the user can successively apply to the actuator lever 520 with his or her hand at a time. That is, it is possible to couple the medical tube fixing device 300 with the medical tube 20 and fix the medical tube fixing device 300 to the skin while pressing the actuator lever 520 by a single-sequence pressing operation.

FIG. 16 shows a state in which the pressure applied to the actuator lever 520 of the skin stapler 500 is removed. When the user completely removes the force pressing the actuator lever 520 in the state where the medical tube 20 has been fixed to the skin, a restoring force of the elastic member 580 causes the actuator lever 520 to be returned to its original position. As a result, the first and second pressing parts 565 and 575 are also retracted, so that the holding of the medical tube 20 by the medical tube fixing device 300 may be released.

It is necessary to quickly and easily carry out the task of fixing the medical tube 20 in the field of operations. If a surgery takes a lot of time to finish an operation, it may increase the probability that the patient will become infected with various germs. The medical tube fixing devices 10 and 300 according to the embodiments of the present invention include the body unit 100 or 310 that can enclose and hold the medical tube 20, and the fixing pins 200 or 350 that are used to be fixed to the patient's skin. The body units 100 or 310 and the fixing pins 200 or 350 are coupled to be a single body structure that can be easily and quickly mounted on the skin stapler 500. With the simple single-sequence of pressing operation that the user pushes up the actuator lever 520 of the skin stapler 500 to apply the first pressure and the second pressure, the medical tube fixing device 10 or 300 mounted on the skin stapler 500 can be coupled with medical tube 20 and fixed to the skin. Therefore, the fixing operation of the medical tube 20 can be performed very quickly and easily. Using the medical tube fixing device 10 or 300 and the skin stapler 500 is convenient and can reduce the infection rate at the surgical site.

The foregoing is illustrative of example embodiments and is not to be construed as limiting thereof. Although a few example embodiments have been described, those skilled in the art will readily appreciate that many modifications are possible in the example embodiments without materially departing from the novel teachings and advantages of the present inventive concept. Accordingly, all such modifications are intended to be included within the scope of the present inventive concept as defined in the claims. Therefore, it is to be understood that the foregoing is illustrative of various example embodiments and is not to be construed as limited to the specific example embodiments disclosed, and that modifications to the disclosed example embodiments, as well as other example embodiments, are intended to be included within the scope of the appended claims.

What is claimed is:

1. A medical tube fixing device, comprising:
a body unit configured to include first and second fixing members which are coupled to each other so as to be openable and closable and capable of enclosing and holding an outer surface of a medical tube in a hollow cylindrical shape while facing each other when being closed by a first external force; a non-slip member provided on an inner surface of at least one of the first and second fixing members and configured to enhance a frictional force with the outer surface of the medical tube; and a locking unit configured to lock the first and second fixing members to each other so that the first fixing member and the second fixing member do not open in a closed state; and
at least a pair of fixing pins configured to protrude outwardly from the body unit while being fixed to the body unit, to have a shape bent in a direction in which end parts thereof are closer to each other so that the pair of fixing pins are further bent by a second external force pressing them by a skin stapler to form a closed loop when end parts thereof come to meet each other.

2. The medical tube fixing device according to claim 1, wherein the at least one pair of fixing pins are configured to extend in both opposite directions substantially perpendicular to an axial direction of the body unit to provide a protruding portion outside the body unit, so that the at least one pair of fixing pins can receive the second external force directly from the skin stapler.

3. The medical tube fixing device according to claim 1, wherein the medical tube fixing device is intended to be used for a purpose that the first and second fixing members of the body unit are closed to enclose and hold the medical tube by a pressure successively applied by the skin stapler in a state that the medical tube fixing device is loaded on the skin stapler.

4. The medical tube fixing device according to claim 1, wherein the body unit is configured to have an inner diameter smaller than an outer diameter of the medical tube and to be in close contact with the outer surface of the medical tube.

5. The medical tube fixing device according to claim 1, wherein the locking unit comprises a first supporting plate formed with a through-hole and fixed to the first fixing member; and a second supporting plate formed with a protrusion to be press-fitted into the through-hole and fixed to the second fixing member.

6. The medical tube fixing device according to claim 1, wherein the pair of fixing pins are fixed to the first supporting plate and the second supporting plate, respectively.

7. The medical tube fixing device according to claim 1, wherein the non-slip member is any one of a plurality of elastic protrusions or elastic non-slip pads coated on at least a part of an inner surface of at least one of the first fixing member and the second fixing member.

8. A skin stapler, comprising:
a housing;
an elastic member installed inside the housing;
an actuator lever configured to be a structure that a part of the actuator lever is exposed to an outside of the housing and the other part of the actuator lever is present inside the housing, and to be supported and pushed out of the housing by the elastic member while being caught by the housing so as not to escape to the outside of the housing;
a body tightening unit disposed outside the housing and configured to include a first gripping hand structure which is able to be pivotally closed around a first axis so as to enclose and apply a first force to an outer surfaces of an openable cylindrical body unit of a medical tube fixing device to be pinched and closed;
a fixing pin tightening unit disposed outside the housing and configured to include a second gripping hand structure which is able to be pivotally closed around a second axis so as to enclose and apply a second force to at least a pair of fixing pins of the medical tube fixing device so that distal ends of the pair of fixing pins come close to each other;
a first pressure transmission mechanism built in the housing and configured to receive a first pressure from the actuator lever and to convert the first pressure into the first force to pinch the body tightening unit and transmit the first force to the body tightening unit; and
a second pressure transmission mechanism built in the housing and configured to receive a second pressure from the actuator lever and to convert the second pressure into the second force to pinch the fixing pin tightening unit and transmit the second force to the fixing pin tightening unit.

9. The skin stapler according to claim 8, wherein the skin stapler is intended to be used in a manner that, when the body unit and the pair of fixing pins are respectively loaded on the body tightening unit and the fixing pin tightening unit, a force applied by a user to the actuator lever of the skin stapler is transferred to the body tightening unit and the fixing pin tightening unit to pinch the body unit and the pair of fixing pins for a purpose of coupling the medical tube fixing device with a medical tube and fixing the medical tube fixing device to skin.

10. The skin stapler according to claim 8, wherein the first pressure transmission mechanism comprises a first pressing part provided at a distal end thereof and configured to press the body tightening unit to be pinched while being advanced by the first pressure.

11. The skin stapler according to claim 8, wherein the second pressure transmission mechanism comprises a second pressing part provided at a distal end thereof and configured to press the fixing pin tightening unit to be pinched while being advanced by the second pressure.

12. A medical tube fixing apparatus, comprising:
a medical tube fixing device; and
a skin stapler,
wherein the medical tube fixing device comprises:
a body unit configured to include first and second fixing members which are coupled to each other so as to be openable and closable and capable of enclosing and holding an outer surface of a medical tube in a hollow cylindrical shape while facing each other when being closed by a first external force; a non-slip member provided on an inner surface of at least one of the first and second fixing members and configured to enhance a frictional force with the outer surface of the medical tube; and a locking unit configured to lock the first and second fixing members to each other so that the first fixing member and the second fixing member do not open in a closed state; and
at least a pair of fixing pins configured to protrude outwardly from the body unit while being fixed to the body unit, to have a shape bent in a direction in which end parts thereof are closer to each other so that the pair of fixing pins are further bent by a second external force pressing them by a skin stapler to form a closed loop when end parts thereof come to meet each other,
wherein the skin stapler comprises:
a housing;
an elastic member installed inside the housing;
an actuator lever configured to be a structure that a part of the actuator lever is exposed to an outside of the housing and the other part of the actuator lever is present inside the housing, and to be supported and pushed out of the housing by the elastic member while being caught by the housing so as not to escape to the outside of the housing;
a body tightening unit disposed outside the housing and configured to include a first gripping hand structure which is able to be pivotally closed around a first axis so as to enclose and apply a first force to an outer surfaces of an openable cylindrical body unit of a medical tube fixing device to be pinched and closed;
a fixing pin tightening unit disposed outside the housing and configured to include a second gripping hand structure which is able to be pivotally closed around a second axis so as to enclose and apply a second force to at least a pair of fixing pins of the medical tube fixing device so that distal ends of the pair of fixing pins come close to each other;
a first pressure transmission mechanism built in the housing and configured to receive a first pressure from the actuator lever and to convert the first pressure into the first force to pinch the body tightening unit and transmit the first force to the body tightening unit; and
a second pressure transmission mechanism built in the housing and configured to receive a second pressure from the actuator lever and to convert the second pressure into the second force to pinch the fixing pin tightening unit and transmit the second force to the fixing pin tightening unit, and
wherein the medical tube fixing apparatus is intended to be used in a manner that, when the medical tube fixing device id loaded on the skin stapler, the skin stapler presses the body unit and the pair of fixing pins by the body tightening unit and the fixing pin tightening unit to pinch the body unit and the pair of fixing pins for a purpose of coupling the medical tube fixing device with a medical tube and fixing the medical tube fixing device to skin.

13. The medical tube fixing apparatus according to claim 12, wherein the non-slip member is any one of a plurality of elastic protrusions or elastic non-slip pads coated on at least a part of an inner surface of at least one of the first fixing member and the second fixing member.

14. The medical tube fixing apparatus according to claim 12, wherein the at least one pair of fixing pins are configured to extend in both opposite directions substantially perpendicular to an axial direction of the body unit to provide a protruding portion outside the body unit, so that the at least one pair of fixing pins can receive the second external force directly from the skin stapler.

15. The medical tube fixing apparatus according to claim 12, wherein the first pressure transmission mechanism comprises a first pressing part provided at a distal end thereof and configured to press the body tightening unit to be pinched while being advanced by the first pressure.

16. The medical tube fixing apparatus according to claim 12, wherein the second pressure transmission mechanism comprises a second pressing part provided at a distal end thereof and configured to press the fixing pin tightening unit to be pinched while being advanced by the second pressure.
